(19) United States
(12) Patent Application Publication
(10) Pub. No.: US 2008/0014153 A1
(43) Pub. Date: Jan. 17, 2008

(54) Topical Preparation For Application To The Skin Containing Natural Oil Of The Evening Primrose (Oenothera Biennis) (=Oleum Oenotherae) And Osmolytes Originating From Extremophilic Microorganisms

(76) Inventor: Thomas Schwarz, Leichlingen (DE)

Correspondence Address: BERLINER & ASSOCIATES, 555 WEST FIFTH STREET, 31ST STREET, LOS ANGELES, CA, 90013, US

(21) Appl. No.: 10/594,576

(22) PCT Filed: Mar. 30, 2005
(86) PCT No.: PCT/EP05/03342

§ 371 Date: Jun. 28, 2007

(30) Foreign Application Priority Data
Mar. 30, 2004 (DE) 10 2004 016 045.7
Oct. 8, 2004 (DE) 10 2004 049 062.7

(51) Int. Cl. A61K 36/185; A61K 35/66; A61P 17/00

(52) U.S. Cl. 424/59; 424/520; 424/776

(57) ABSTRACT

The invention relates to formulations comprising natural oils and fats that contain an osmolyte originating from extremophilic microorganisms and unsaturated fatty acids, with the osmolyte being present in an amount ranging between 0.01 and 50 wt. % based on the total weight of the formulation. The invention also relates to the use of osmolytes originating from extremophilic microorganisms for the stabilization of unsaturated fatty acids. Especially preferred is a combination of osmolyte and evening primrose oil (Oleum Oenotherae).

The invention relates to a formulation comprising natural oils and fats containing an osmolyte originating from extremophilic microorganisms and unsaturated fatty acids. The formulation contains at least one osmolyte in concentrations ranging from 0.01 to 50 wt. % based on the total weight of the formulation.

In particular, the invention relates to the use of an osmolyte obtained from extremophilic bacteria (e.g. ectoine, hydroxyectoine, firoin, firoin-A, diglycerol phosphate, cyclic diphosphoglycerate, diinositol phosphate, hereinafter referred to as osmolytes X) in combination with natural evening primrose oil (Oleum Oenotherae) for the production of medicine, medical products or cosmetic formulations for the external treatment, prophylaxis or care of the skin.

The human organism essentially needs certain fatty acids which have to be ingested with food substances in order to avoid deficiency symptoms. This includes polyunsaturated fatty acids (PUFAs) of the omega-6 and omega-3 series such as, for example, linoleic acid and alpha linoleic acid which, being precursors of eicosanoids, influence numerous physiological functions.

Also the stability of the fats is very much dependent on the PUFA concentration. In this context the influence of oxygen, light and heat may result in the formation of hydroperoxides. These highly reactive molecules may then form polymers (resinification, drying up) and low-molecular fragments causing a rancid odor. Vitamin E, for example, improves the resistance to oxidative changes. Therefore, only relatively stable oils such as, for example, olive or groundnut oils should be used for cooking or frying purposes.

Unsaturated fatty acids may further be categorized into monounsaturated and polyunsaturated fatty acids (PUFA). Naturally occurring unsaturated fatty acids usually have a cis-configuration at the double bonds. So-called trans fats are produced through “hardening” of fat during which the double bonds of unsaturated fatty acids undergo hydrogenation resulting in the formation of vegetable fats that are also hard at room temperature. In animal fat and in the first stomach of ruminants linoleic acid derivatives are produced through hydrogenation that contain two conjugated double bonds of cis- and/or trans-configuration and are referred to as conjugated linoleic acid (CLA).

| Scientific Name | Common Name | Short Designation |
|---|---|---|
| 5-dodecenoic acid | Lauroleinic acid | 12:1 (5) |
| 9-Tetradecenoic acid | Myristoleic acid | 14:1 (9) |
| 9-Hexadecenoic acid | Palmitoleic acid | 16:1 (9) |
| 9-Octadecenoic acid | Oleic acid | 18:1 (9) |
| 9-Eicosenoic acid | Gadoleic acid | 20:1 (9) |
| 13-Docosenoic acid | Erucic acid | 22:1 (13) |
| 9,12-Octadecadienoic acid | Linoleic acid | 18:2 (9,12) or 18:2, |
| | | Omega-6 |
| 9,12,15-Octadecatrienoic acid | alpha linoleic acid | 18:3 (9,12,15) or 18:3, |
| | | Omega-3 |
| 6,9,12-Octadecatrienoic acid | y-linoleic acid | 18:3 (6,9,12) or 18:3, |
| | | Omega-6 |
| 5,8,11,14-Eicosatetraenoic | Arachidonic acid | 20:4, |
| acid | | Omega-6 |
| 5,8,11,14,17- | | 20:5, |
| Eicosapentaenoic acid | | Omega-3 |
| 4,7,10,13,16,19- | | 22:6, Omega-3 |
| Docosahexaenoic acid | | |

All mono- and polyunsaturated fatty acids are more or less instable with respect to the influence of oxidation and therefore have to be stabilized by low temperatures or suitable substances unless they are meant to be used immediately. The same applies of course to compositions and formulations containing unsaturated fatty acids, especially natural oils and fats.

Osmolytes or compatible solutes (see above) from extremophilic microorganisms constitute a known group of low-molecular protective substances. Extremophiles are rather extraordinary microorganisms because they grow optimally, for instance, at high salt concentrations (up to 200 g NaCl/l) and elevated temperatures (60-110° C.) that in the event of mesophilic (“normal”) organisms would lead to an extensive damage of cellular structures. In recent years comprehensive research efforts have been made to identify the biochemical components that bring about the remarkable thermal, chemical and physical stabilization of the cell structures. Although many enzymes from hyperthermophilic microorganisms are stable even under elevated temperatures this is not generally true for the cellular structures of thermophilic and hyperthermophilic organisms. The high temperature stability of cell structures is—to a remarkable extent—due to low-molecular organic substances (compatible solutes, osmolytes) present in the intracellular environment. In recent years various novel osmolytes could be identified in extremophilic microorganisms for the first time. In some cases it could be shown that these compounds effectively contributed to the protection of cellular structures—first of all enzymes—against heat and dryness. (Lippert, K., Galinski, E. A. (1994), Appl. Microbiol. Biotech. 37, 61-65; Louis, P., Trüper, H. G., Galinski, E. A. (1994), Appl. Microbiol. Biotech. 41, 684-688; Ramos, Raven, Sharp, Bartolucci, Rossi, Cannio, Lebbink, v.d. Oost, de Vos, Santos (1997), Appl. Environm. Microbiol. 63, 4020-4025; Da Costa, Santos, Galinski (1998), Adv. In Biochemical Engineering Biotechnology, 61, 117-153).

Osmolytes (compatible solutes) found in extremophilic microorganisms are not produced by human or animal cells.

Processes developed by bitop AG (Method for the in-vivo isolation of cell constituents; EP 94 903 874.9; Method for the isolation of valuable substances of organisms through influencing/impairing the cell-inherent transporting systems of these valuable substances; EP 98 121 243.4; Method for the separation and isolation of highly pure, low-molecular, structurally similar compounds, in particular tetrahydropyrimidin derivatives; DE10047444.6) enable osmolytes of highest play an important role for the metabolism, particularly with respect to the regeneration of cells.

The cold-extracted evening primrose oil (Oleum Oenotherae) contains important amino acids, minerals as well as vitamins including vitamin E functioning as a natural antioxidizing substance. The oil is not only used in high-grade cosmetic formulations but is also considered a valuable dietary supplement. The tending effect of the evening primrose oil has been pointed out in numerous cosmetic products when very dry, irritated, scaly and problematic skin has to be treated. Moreover, evening primrose oil may also be ingested in the form of a dietary supplement.

Storage and application of evening primrose oil (Oleum Oenotherae) in topical preparations is rather limited as a result of its poor durability. Since evening primrose oil is an easily perishable product it is usually preserved by adding vitamin E. Furthermore, evening primrose oil is always mixed with another base oil to improve its stability. Through these measures the oil's durability is improved significantly. It is to be noted that due to its instability evening primrose oil cannot be heated, for instance as a preservation method.

The same applies to a number of other natural oils that to a higher or lesser degree have been commonly employed in the foodstuff, cosmetics and pharmaceutical industry, including fish oil (e.g. mackerel, tuna, salmon, herring), coconut oil, palm oil, cocoa oil, olive oil, groundnut oil, cotton seed oil, sesame oil, corn oil, safflower oil, soy bean oil, wheat oil, borage oil, linseed oil, rapeseed oil, black hempnettle oil, castor oil, nigella oil, dogrose kernel oil, hempseed oil and the like.

Surprisingly, it has now been determined that adding osmolytes X of extremophilic microorganisms to the natural oils of Oenothera biennis (=Oleum Oenotherae) in formulations for the topical (superficial) application to the skin significantly improves the stability of the evening primrose oil in the topical preparation intended for skin treatment. Through the addition of osmolytes X the stability of and thus preservation effect on the evening primrose oil can thus be improved distinctly without limiting the range of application within cosmetic and derma-quality to be obtained in sufficient amount from extremophilic microorganisms. Using these substances as pharmaceutical preparations, cosmetic formulations or medical products is thus conceivable on principle.

The osmolyte ectoine has hitherto been used as moisturizer and active constituent of cosmetic preparations with a view to protecting healthy, clinically inconspicuous human skin against the detrimental effects of ultraviolet solar radiation (e.g. Use of ectoine or ectoine derivatives in cosmetic formulations, EP19990941; Use of ectoine or ectoine derivatives for the prophylaxis and/or treatment of UV induced immunosuppression). In this context in-vitro and in-vivo investigations have shown that this cosmetic effect of ectoine is based, inter alia, on the fact that this molecule influences the function of epidermal Langerhans cells as well as epidermal keratinozytes and dermal fibroblasts in such a manner that these cells are better protected against the proinflammatory effects of UV radiation which, for example, would give rise to sunburns.

The production of a pharmaceutical preparation intended for the treatment of skin diseases by means of ectoine or hydroxyectoine is described generally by the European Patent Specification EP 98 113 132.9 taken out by bitop AG.

The evening primrose (Oenothera biennis; plant family Onagraceae), also called ((Queen of the Night)), is a plant that opens its bright yellow blossoms when the weather is dull or in the evening, hence the name evening primrose. More than 500 years ago in North America where the evening primrose has originated Algonquin Indians used to treat various skin diseases by applying a paste of pestled evening primrose seeds (Oleum Oenotherae).

The oil obtained from the seeds of the evening primrose (Oleum Oenotherae) contains a high concentration of polyunsaturated fatty acids such a linoleic acid and gammalinolenic acid. The seeds employed for the production of the oil stem from plants grown without making use of agricultural control chemicals. The evening primrose oil is a natural and biologically active source of essential fatty acids including, inter alia, cis-gammalinolenic acid (GLA) in a proportion of approx. 10%. The activity of the acid is due to its unique trigliceryde structure which is exclusively found in the evening primrose seed oil. These fatty acids tological products. Moreover, the temperature stability of evening primrose oil (Oleum Oenotherae) is significantly enhanced by the addition of osmolytes which enables evening primrose oil to be thermally preserved without its natural properties being lost.

Furthermore, another surprising effect was discovered in that an evening primrose oil containing product to which 0.5% (wt. percent) of the osmolyte ectoine had been added resulted in a noticeably better tending effect on very dry, irritated and problematic skin than was observed with an evening primrose oil containing tending product of identical composition. The tending effect of the product combination of evening primrose oil and osmolyte was, surprisingly, found to be significantly better than with a care product just containing the osmolyte of 0.5% concentration.

Therefore, a preparation to be applied topically containing a combination of evening primrose oil (Oleum Oenotherae) and osmolytes X for cosmetic and dermatological applications surprisingly offered distinct advantages in terms of improved preservation and longer durability and at the same time had a more efficient tending effect when treating very dry, irritated, scaly and problematic skin. In this way a preparation combining osmolytes and evening primrose oil is also ideally suited for the tending and care of inflamed skin areas found with people suffering, for example, from atopic dermatitis.

The cosmetic and dermatological preparations are produced in that one or several compounds of the group of osmolytes, the physiologically compatible salts of compounds of the osmolytes in combination with the oil (Oleum Oenotherae) of the evening primrose (Oenothera biennis), if expedient with the aid of auxiliary and/or carrier substances, are appropriately dealt with so as to bring about a suitable formulation.

The auxiliary and carrier substances stem from the group of carrier agents, preservative agents and other customary auxiliary agents. The osmolytes and the evening primrose oil contained in cosmetic and dermatological formulations are to be applied topically. They may, for example, be used in the form of solutions, suspensions, emulsions, pastes, ointments, gels, creams, lotions, powder, soaps, surfactant-containing cleaning preparations, oils and sprays. Moreover, customary carrier substances, auxiliary substances of any kind and, if thought expedient, further agents may be added to the formulations. Preferable auxiliary substances stem from the group of preservative agents, antioxidants, stabilizers, solutizers, vitamins, coloring agents and de-odorizers.

Aside from one or several compounds selected from the group of osmolytes plus evening primrose oil ointments, pastes, creams and gels may contain customary carrier substances such as, for example, animal and vegetable fats, waxes, paraffins, starch, traganth, cellulose derivatives, polyethylene glycols, silicones, bentonites, silicic acid, talcum and zinc oxide or mixtures/blends of these substances.

Aside from one or several compounds selected from the group of osmolytes plus evening primrose oil powders and sprays may contain in addition to the customary carrier substances customary propellants, for instance chlorofluorohydrocarbons, propane/butane or dimethyl ether.

Aside from one or several compounds selected from the group of osmolytes X plus evening primrose oil (Oleum Oenotherae) solutions and emulsions may contain customary carrier substances such as solvents, solutizers and emulgators.

Aside from one or several compounds selected from the group of osmolytes X plus evening primrose oil (Oleum Oenotherae) suspensions may contain additional carrier substances such as, for example, water or ethanol.

Further forms of application are, for instance, soaps, surfactant-containing cleansing agents, face and body oils, lipsticks, lip-care sticks, mascara, eyeliners, eye shadowing, rouge, powder, emulsion and wax make-up, as well as sunscreen, pre-sun and after-sun preparations.

The proportion of the compounds of the group of osmolytes X in cosmetic and dermatological formulations preferably amounts to 0.0001 to 50 wt. %, especially preferred is 0.001 to 10 wt. % based on the entire formulation.

The proportion of the evening primrose oil (Oleum Oenotherae) in cosmetic and dermatological formulations preferably amounts to 0.0001 to 50 wt. %, especially preferred is 0.001 to 10 wt. % based on the entire formulation.

The following examples are meant to provide elucidation on the present invention but shall by no means limit its scope.

### EXAMPLE 1

### O/W Lotion

According to the invention an osmolytes X and evening primrose oil (Oleum Oenotherae) containing lotion (O/W) is made from the following components:

| | Percentage |
|---|---|
| Components | by Weight (% w/v) |
| Evening primrose oil (Oleum | 8.00 |
| Oenotherae) | |
| Isopropyl palmitate | 3.00 |
| Petrofatum | 4.00 |
| Cetylstearyl alcohol | 2.00 |
| Sodium cetylstearyl sulfate | 0.50 |
| Sodium carbomer | 0.40 |
| Osmolyte ectoine | 0.50 |
| Octylmethooxycinnamate | 5.00 |
| Butylmethoxydibenzoylmethane | 1.00 |
| Water | Ad 100.00 |

### EXAMPLE 2

### O/W Cream

According to the invention an osmolyte X containing cream (O/W) is made from the following components:

| | Percentage |
|---|---|
| Components | by Weight (% w/v) |
| Evening primrose oil (Oleum Oenotherae) | 10.00 |
| Glycerylmonostearate | 4.00 |
| Osmolyte X | 0.50 |
| Titanium dioxide | 1.00 |
| Sodium lactate | 3.00 |
| Water | Ad 100.00 |

### EXAMPLE 3

### O/W Cream with Cyclic Diphosphoglycerate (cDPG)

According to the invention an osmolyte cyclic diphosphoglycerate (cDPG)-containing cream (O/W) is made from the following components:

| | Percentage |
|---|---|
| Components | by Weight (% w/v) |
| Evening primrose oil (Oleum Oenotherae) | 10.00 |
| Glycerylmonostearate | 4.00 |
| Osmolyte cDPG | 0.50 |
| Titanium dioxide | 1.00 |
| Sodium lactate | 3.00 |
| Water | Ad 100.00 |

### EXAMPLE 4

### Stabilization of a Solution Consisting of γ-linolenic Acid Obtained from Evening Primrose Plants Using the Osmolyte Diglycerol Phosphate (DGP)

The stability of evening primrose oil at higher temperatures (room temperature, 25° C.) is largely dependent on the γ-linolenic acid content and the purity of the evening primrose oil. After 0.1% (wt. percent) of diglycerol phosphate had been added to a preparation of evening primrose oil an improved evening primrose oil storage stability of five days was noticed compared to a check preparation that did not contain diglycerol phosphates. The stability of the preparation was assessed based on the first occurrence of a rancid smell of the evening primrose oil.

### EXAMPLE 5

### Improved Temperature Stability of Evening Primrose Oil When an Osmolyte Blend of Mannosylglycerate (Firoin) and Mannosylglyceramide (Firoin-A) Was Added

Increasing the temperature to 70° C. within a period of one hour causes evening primrose oil to become quickly destabilized/denaturized with hydroperoxides forming. These highly reactive molecules may then form polymers (resinification, drying up) and low-molecular fragments causing a rancid odor. A 50:50 blend of the osmolytes mannosylglycerate and mannosylglyceramide of a 5 wt. % concentration enhances the resistance of evening primrose oil to oxidative changes at temperatures of 70° C. by a time span of 24 hours. In the presence of a 50-% mannosylglycerate-mannosylglyceramide mixture a rancid odor can only be noticed after a 24-hour incubation period at 70° C. The use of a mannosylglycerate-mannosylglyceramide mixture can thus significantly improve, for example, the processing and storage properties of evening primrose oil and of products containing evening primrose oil.

### EXAMPLE 6

### Gel Containing Evening Primrose Oil and the Osmolyte Diinositol Phosphate

According to the invention a gel (O/W) containing diinositol phosphate and evening primrose oil is made from the following components:

| | Percentage |
|---|---|
| Components | by Weight (% w/v) |
| Carbopol | 2.00 |
| Triethanolamine | 3.00 |
| Di-inositol phosphate | 0.50 |
| Evening primrose oil | 10.00 |
| Tocopherylacetate | 0.20 |
| Polyoxyethylene sorbitan fatty acid ester | 0.50 |
| (Tween 20) | |
| Glycerine | 2.00 |
| Sodium PCA | 0.50 |
| Hydrolized collagen | 2.00 |
| Preservative agents, coloring matter, | q.s. |
| perfume | |
| Water | Ad 100.00 |

As preservative agent 0.05% propylhydroxybenzoate or 0.15% methyl-4-hydroxybenzoate may be used.

### EXAMPLE 7

### Sunscreen Emulsion Containing Evening Primrose Oil and the Osmolyte Hydroxyectoine

According to the invention a sunscreen emulsion containing evening primrose oil and hydroxectoine is made from the following components:

| | Percentage by |
|---|---|
| Components | Weight (% w/v) |
| Cyclomethicone | 2.00 |
| Evening primrose oil | 10% |
| Cetyldimethicone copolyol | 0.20 |
| PEG 22 dodecyl copolymer | 3.00 |
| Paraffin oil (DAB 9) | 2.00 |
| Caprylic acid/Caprinic acid triglyceride | 5.80 |
| Octylmethoxycinnamate | 5.80 |
| Butyl methoxy dibenzoylmethane | 4.00 |
| Tocopherylacetate | 0.50 |
| ZnSO4 | 0.70 |
| Na3IIEDTA | 0.30 |
| Hydroxyectoine | 2.50 |
| Preservative agents, coloring matter, perfume | q.s. |
| Water | ad 100.00 |

1. Formulation comprising natural oils and fats containing an osmolyte originating from extremophilic microorganisms and unsaturated fatty acids, characterized in that said formulation contains 0.01 to 50 wt. % of at least one osmolyte based on the total weight of the formulation.

2. Formulation according to claim 1, characterized in that said formulation contains 0.05 to 10 wt. %, particularly 0.1 to 5 wt. % of an osmolyte or of osmolytes.

3. Formulation according to claim 1 or 2, characterized in that it contains 0.01 to 50 wt. % of natural oils and fats.

4. Formulation according to claim 3, characterized in that said formulation contains 0.05 to 10 wt. %, particularly 0.1 to 50 wt. % of natural oils an fats.

5. Formulation according to at least one of the above claims, characterized in that it contains evening primrose oil (Oleum Oenotherae).

6. Formulation according to at least one of the above claims, characterized in that ectoine, hydroxyectoine, cDPG, DGP, firoin, firoin A and/or diinositol phosphate are used as osmolyte.

7. Formulation according to any one of the claims 1 to 6 for cosmetic application to the skin.

8. Cosmetic formulation according to claim 7, characterized in that it is provided in the form of a solution, a suspension, an emulsion, a paste, an ointment, a gel, a cream, a lotion, a powder, a soap, a surfactant-containing cleansing agent, an oil, a lipstick, a lip-care stick, a mascara, an eyeliner, of eye shadowing, rouge, a powder, emulsion and wax make-up, a sunscreen, pre-sun and after-sun preparation, a hair tonic, a plaster, a bandage or spray.

9. Use of an osmolyte originating from extremophilic microorganisms for the stabilization and/or preservation of unsaturated fatty acids.

10. Use according to claim 9, characterized in that the applied osmolyte is present in an amount of between 0.01 and 50 wt. % in relation to the entire formulation.

11. Use according to claim 9 or 10, characterized in that it contains 0.05 to 10 wt. %, particularly 0.1 to 5 wt. % of an osmolyte or of osmolytes.

12. Use according to at least one of the claims 9 to 11, characterized in that ectoine, hydroxyectoine, cDPG, DGP, firoin, firoin A and/or diinositol phosphate are used as osmolyte.

13. Use according to at least one of the claims 9 to 12 for the stabilization of evening primrose oil (Oleum Oenotherae).

14. Use of an osmolyte originating from extremophilic microorganisms for the production of formulations for topical applications in the cosmetic and dermatological field.

15. Use according to claim 14 for the production of cosmetic formulations for the tending and care of very dry, irritated, scaly and problematic skin, for the tending and care of inflamed skin in case of atopic dermatitis, psoriasis and other inflammable skin diseases, for the protection and stabilization of human skin cells against physical, chemical and biological influences, in particular UV and IR radiation and denaturizing substances, for the protection of the skin's microflora, for the stabilization of the natural skin barrier and as free-radical scavenger and/or antioxidants.

16. Use according to claim 14 for the production of medicinal products and/or medicaments for the tending, care, prophylaxis or treatment of atopic dermatitis, psoriasis, other inflammable skin diseases as well as eczema.

17. Use according to any one of claims 14 to 16, characterized in that the osmolyte is ectoine or hydroxyectoine.

18. Use according to at least one of the claims 14 to 17 in the form of a solution, a suspension, an emulsion, a paste, an ointment, a gel, a cream, a lotion, a powder, a soap, a surfactant-containing cleansing agent, an oil, a lipstick, a lip-care stick, a mascara, an eyeliner, of eye shadowing, rouge, a powder, emulsion or wax make-up, a sunscreen, pre-sun and after-sun preparation, a hair tonic, a plaster, a bandage or spray.

19. Use according to claim 18, characterized in that the formulation further contains at least one UV filter, enzymes, vitamins, vitamin derivatives and/or proteins.
